(19) United States
(12) Patent Application Publication
(10) Pub. No.: US 2025/0033042 A1
(43) Pub. Date: Jan. 30, 2025

(54) MICROFLUIDIC DEVICES WITH DIELECTROPHORETIC ACTUATORS

(71) Applicant: Hewlett-Packard Development Company, L.P., Spring, TX (US)

(72) Inventors: Viktor SHKOLNIKOV, Palo Alto, CA (US); Angela BAKKOM, Corvallis, OR (US); Christopher Hans BAKKER, Corvallis, OR (US); Matthew David SMITH, Corvallis, OR (US)

(73) Assignee: Hewlett-Packard Development Company, L.P., Spring, TX (US)

(21) Appl. No.: 18/715,100

(22) PCT Filed: Dec. 10, 2021
(86) PCT No.: PCT/US2021/062895

§ 371 Date: May 30, 2024

(51) Int. Cl. B01L 3/00; G01N 15/02

(52) U.S. Cl. CPC B01L 3/502715; G01N 15/0266; B01L 2200/027; B01L 2300/0645; B01L 2400/0424

(57) ABSTRACT

An example microfluidic device comprises a microfluidic channel fluidically coupled to a reservoir containing a fluid, a first sensor disposed within the microfluidic channel, a second sensor disposed within the microfluidic channel, a first dielectrophoretic (DEP) actuator disposed within the microfluidic channel between the first sensor and the second sensor, and a fluid ejection device fluidically coupled to the microfluidic channel.

### BACKGROUND

Microfluidic systems may be used to perform operations on fluids, such as the manipulation of fluid droplets to facilitate the handling and testing of fluids on a small scale. Such devices may be used in the medical industry, for example to analyze cells, analyze deoxyribonucleic acid (DNA), detect pathogens, perform clinical diagnostic testing, and/or for synthetic chemistry, among other types of industries and/or purposes.

### BRIEF DESCRIPTION OF FIGURES

FIGS. 1A-1B illustrate an example microfluidic device with a dielectrophoretic (DEP) actuator, consistent with the present disclosure.

FIGS. 2A-2D illustrate example operations of a microfluidic device with a DEP actuator, consistent with the present disclosure.

FIGS. 3A-3G illustrate further example microfluidic devices with DEP actuators, consistent with the present disclosure.

FIG. 4 illustrates an example apparatus including sensor circuitry and a controller, consistent with the present disclosure.

FIGS. 5A-5C illustrate other example apparatuses including sensor circuitry and a controller, consistent with the present disclosure.

FIGS. 6A-6B illustrate other example apparatuses including a microfluidic device, sensor circuitry, and a controller, consistent with the present disclosure.

FIG. 7 illustrates an example method of selectively ejecting fluid using a microfluidic device, consistent with the present disclosure.

FIG. 8 illustrates an example microfluidic device with a plurality of microfluidic channels and a plurality of interrogation regions, consistent with the present disclosure.

FIG. 9 illustrates an example microfluidic device with a microfluidic channel and a plurality of interrogation regions, consistent with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

The life sciences research and diagnostics industries are under pressure to reduce costs, increase throughput, and improve the utilization of samples. As a result, the instruments and tools used therein are moving from complex macrofluidic-based systems to simpler microfluidic-based technology, moving from pipetting-based technology to dispensing-based technology, and moving from performing a single test per sample to performing multiplexed tests per sample. In some examples, an inkjet-based fluid dispensing devices may be used to perform a test using a microfluidic device. Inkjet-based fluid dispensing devices may start with microliters of fluid and then dispense picoliters or nanoliters of fluid into specific regions on a substrate from the microfluidic device. These dispense regions may be specific target locations on the substrate surface, such as cavities, microwells, channels, or indentations into the substrate. As used herein, a microwell includes and/or refers to a column capable of storing a volume of fluid between a nanoliter and several milliliters of fluid. There may be tens, hundreds, or even thousands of dispense regions on the substrate, which may represent many tests on a small number of samples, a small number of tests on many samples, or a combination of the two. Additionally, multiple dispensing nozzles or fluid ejection devices (e.g., printheads) may dispense fluid on the substrate at a time to enable a high-throughput design.

In various applications, it may be beneficial to isolate a type of particle or particle population in each of a plurality of regions of the substrate. Different particle populations or n-particles may be useful for different types of tests performed. As used herein, a particle includes and/or refers to a localized object or biologic matter which may have or exhibit particular particle properties, such as size, shape, and dielectric properties. For example, the particles may be dielectric particles. The particles may include cells, nucleic acids, amino acids, antibodies, liposomes, and chemical compounds, among other types of particles and combinations thereof, such as clumps of cells or debris. As a specific example, it may be beneficial to isolate a type of cell from a remainder of particles of a sample. As a further specific example, it may be beneficial to sort and isolate T-cells from other lymphocyte cells for transforming the T-cells to Chimeric antigen receptor (CAR) T-cells for therapy. In some examples, single cells may be isolated from a sample. Such samples may contain a viral or cellular material, including prokaryotic or eukaryotic cells, viruses, bacteriophages, mycoplasmas, protoplasts, and organelles. Example samples may comprise mammalian and non-mammalian animal cells, plant cells, algae including blue-green algae, fungi, bacteria, protozoa, etc. Representative samples include whole blood and blood-derived products such as plasma, serum and buffy coat, urine, feces, cerebrospinal fluid or any other body fluids, tissues, cell cultures, cell suspensions, etc. In such instances, particle may be chosen that have particular traits, and by isolating single particle, such as single cells, producers may ensure the highest purity and potency of the final product. For other example tests, a plurality of particles may be beneficial, such as for antibody tests and/or multiplexed analyses.

In some examples, different types of particles may be sorted and/or isolated based on the particle type. Sorting particles may involve labeling the particles for detection. For example, fluorescently activated cell sorting (FACS) may be used, where particles are labeled with a fluorescent antibody and are interrogated by a laser detector pair. Depending on the fluorescent signal detected by the laser detector pair, the particles may be deflected in a droplet of fluid with an electric field. In other examples, particles may be sorted by magnetic activated particle sorting, where the particles are labeled with an antibody attached to a magnetic bead and magnetic fields are used to isolate particles. Labeling particles involves developing and/or acquiring the specific label, which may increases the costs and complexity for sorting. In many instances, labeled cells or other particles may be incompatible with downstream applications as the act of labeling may change the behavior of the cell or other particle, such as expression levels as the label may activate a pathway.

Examples in accordance with the present disclosure are directed to microfluidic devices, apparatuses, and methods involving detecting particle properties of particles flowing through a microfluidic device using sensor signals obtained prior to and after application of dielectrophoretic (DEP) forces on the particles and ejecting the particles to regions of a substrate based on the particle properties. DEP and/or a DEP force, as used herein, includes and/or refers to a phenomenon and/or a force exerted on a particle when subjected to a non-uniform electric field, which may cause or include movement of the particle in response to the non-uniform electric field. An example microfluidic device comprises a microfluidic channel fluidically coupled to a reservoir containing a fluid, a first sensor disposed within the microfluidic channel, a second sensor disposed within the microfluidic channel, a DEP actuator disposed within the microfluidic channel between the first sensor and the second sensor, and a fluid ejection device fluidically coupled to the microfluidic channel. The fluid ejection device is to actuate to cause flow of the fluid from the reservoir and along the microfluidic channel such that a plurality of particles within the fluid pass over the first sensor, the DEP actuator, and the second sensor, and to eject a volume of the fluid from the microfluidic device. For example, as the particles pass over the first sensor, the DEP actuator, and the second sensor, information may be obtained about the particles and used to determine where to dispense the particles. The information may be used to provide single particle, e.g., cell, sorting without the use of labels.

As used herein, a sample includes and/refers to a volume of fluid containing particles, such as a biologic sample or other fluid including cells and other particles from a biologic sample. Example samples, such as biologic samples, contain any viral or cellular material, including all prokaryotic or eukaryotic cells, viruses, bacteriophages, mycoplasmas, protoplasts, and organelles. Non-limiting examples of a sample includes whole blood and blood-derived products such as plasma, serum and buffy coat, urine, feces, cerebrospinal fluid or any other bodily fluids, tissues, cell cultures, cell suspensions, etc. Non-limiting examples of particles contained in a sample include viral or cellular material, including all prokaryotic or eukaryotic cells, viruses, bacteriophages, mycoplasmas, protoplasts, and organelles, all types of mammalian and non-mammalian animal cells, plant cells, algae including blue-green algae, fungi, bacteria, and protozoa.

Some examples are directed to an apparatus including sensor circuitry and a controller. The sensor circuitry and controller may form part of a fluid dispensing device that uses a microfluidic device to eject fluids to a substrate. The sensor circuitry is to couple to an interrogation region of a microfluidic channel of a microfluidic device and to obtain sensor signals using a first sensor and a second sensor. The interrogation region may include the first sensor, the second sensor, and a DEP actuator disposed between the first sensor and the second sensor. The controller is coupled to the sensor circuitry to cause flow of fluid including a particle through the interrogation region via actuation of a fluid ejection device coupled to the microfluidic channel, apply electric fields within the microfluidic channel via the first sensor, the second sensor, and the DEP actuator, determine a particle property of the particle using the sensor signals received from the sensor circuitry responsive to the flow of the particle through the interrogation region, and cause the fluid ejection device to eject the particle from the microfluidic device to a select region of a substrate based on the determined particle property.

Some examples are directed to a method including flowing fluid containing a plurality of particles from a reservoir to an interrogation region of a microfluidic channel of a microfluidic device, the interrogation region including a first sensor disposed within the microfluidic channel, a second sensor disposed within the microfluidic channel, and a first DEP actuator disposed within the microfluidic channel between the first sensor and the second sensor. While following the fluid through the interrogation region, the method includes applying a first electric field within the microfluidic channel via the first sensor and, in response, obtaining a first sensor signal associated with the plurality of particles using the first sensor, applying a non-uniform electric field within the microfluidic via the DEP actuator, applying a second electric field within the microfluidic channel via the second sensor and, in response, obtaining a second sensor signal associated with the plurality of particles using the second sensor. The method further includes determining particle properties of the plurality of particles using the first sensor signal and the second sensor signal obtained responsive to the flow of the plurality of particles through interrogation region, and selectively ejecting the plurality of particles from the microfluidic device to select regions of a substrate based on the determined particle properties using a fluid ejection device of the microfluidic device.

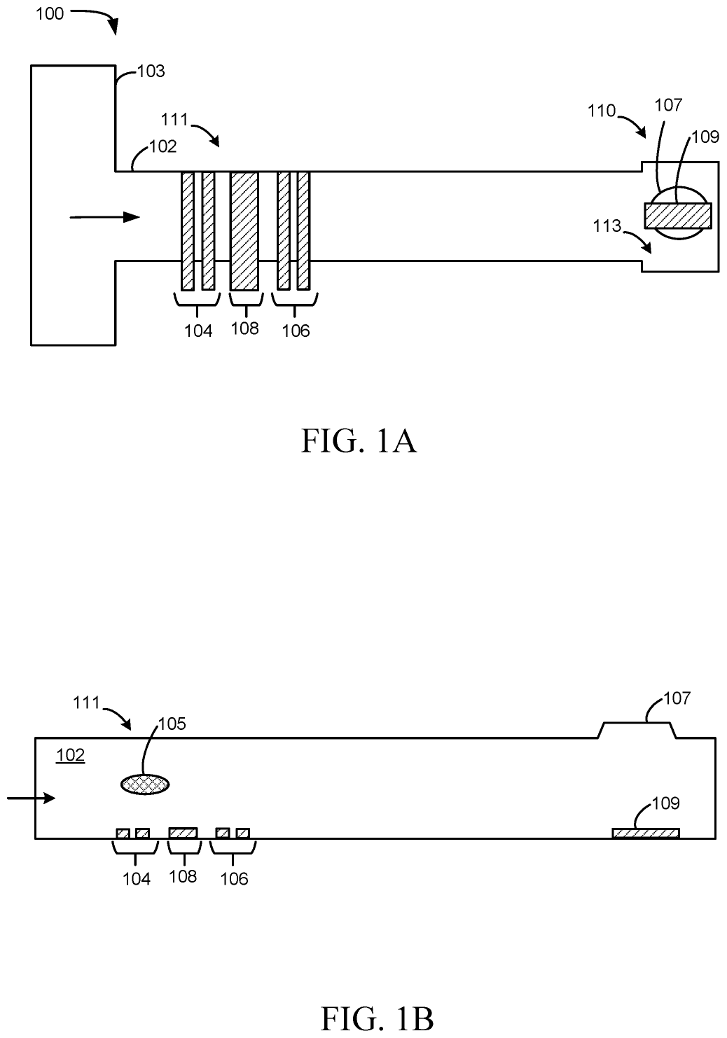

Turning now to the figures, FIGS. 1A-1B illustrate an example microfluidic device with a DEP actuator, consistent with the present disclosure. Microfluidic devices, including the microfluidic device 100 of FIGS. 1A-1B, may be disposable devices used to perform operations on fluid that flows therein, and which may be inserted into and/or disposed within a fluid dispensing device, such as a fluid inkjet device for ejecting fluids to external substrates.

As shown by the top view of the microfluidic device 100 illustrated by FIG. 1A, the microfluidic device 100 includes a microfluidic channel 102 fluidically coupled to a reservoir 103 containing a fluid. The fluid may include a sample fluid containing a plurality of particles. In some examples, the plurality of particles may include different types of cells, clumps of cells, debris, and other cellular and/or molecular material, such as liposomes, ribosomes, membranes, organelles, nucleic acid, proteins, and other material that is intercellular or extracellular. In some examples, the reservoir 103 may form part of the microfluidic device 100 and in other examples, may be separate therefrom and may couple to the microfluidic device 100.

Each of the components of the microfluidic device 100 may be formed on or coupled to a substrate. The substrate may comprise a silicon based wafer or other similar materials used for microfabricated devices (e.g., glass, gallium arsenide, plastics, etc.). In some examples, the microfluidic device 100 may include a housing, such as a cover or lid over the substrate. As further described herein, examples may comprise a microfluidic channel 102 and/or chambers. The microfluidic channel 102 and/or chambers may be formed by etching or micromachining processes in the substrate. Accordingly, the microfluidic channel 102 and/or chambers may be defined by surfaces fabricated in the substrate of the microfluidic device 100.

The microfluidic device 100 further includes a first sensor 104, a second sensor 106, and a DEP actuator 108 disposed within the microfluidic channel 102. As shown by FIG. 1A, the DEP actuator 108 is disposed between the first sensor 104 and the second sensor 106. The first sensor 104 and the second sensor 106 may include a pair of sensing electrodes, and the DEP actuator may include a DEP electrode and a ground electrode. A sensing electrode includes and/or refers to an electrode used to obtain a measurement. In some examples, as further illustrated herein, the first sensor 104, the second sensor 106, and the DEP actuator 108 share a common ground (e.g., a ground pad). The first sensor 104, the second sensor 106, and the DEP actuator 108 may form an interrogation region 111 of the microfluidic device 100. An interrogation region, as used herein, includes and/or refers to a portion of the microfluidic channel 102 used to assess particles within the fluid.

The first sensor 104 and the second sensor 106 may be an impedance-based sensor or another type of sensor, such as an optical sensor, a thermal sensor, a voltammetric sensor, an amperometric/coulometric sensor, a transistor, such as a field-effect transistor, among others. An impedance-based sensor may include a pair of sensing electrodes that measure the impedance or capacitance of the fluid containing the sample, with the capacitance and/or impedance being measured between the electrodes. For example, the impedance or capacitance may be measured for a current or voltage path between the two electrodes. More specifically, a high-frequency alternating (e.g., sine-wave) current or voltage may be applied to one electrode and the interaction of the electric field with the fluid is monitored at the other electrode, which may be in the form of an alternating current signal. The two electrodes may be separated from the fluid by a dielectric layer. Changes in impedance and/or capacitance between the electrodes may indicate the presence of a particle. The impedance measurements may be processed by a controller, as further described herein, to determine a particle property of particles that flow by the first sensor 104 and the second sensor 106. Impedance-based sensors may not contact the particles, which may increase cell viability as compared to sensors that contact the particles, and may be used to sense particles without the use of a label and/or imaging. Additionally, impedance-based sensors may be inexpensive, small in size, and may provide sensor signals at high speeds, as compared to other types of sensors.

The first sensor 104 and the second sensor 106 may be the same type of sensor, or different types of sensors. For instance, the first sensor 104 may be an impedance-based sensor. Similarly, the second sensor 106 may be an impedance-based sensor. Examples of the present disclosure are not limited to impedance-based sensors, and additional and/or different types of sensors may be used.

A DEP actuator 108 includes and/or refers to circuitry and/or a physical structure that causes a DEP force on particles flowing through the microfluidic channel 102. DEP and/or a DEP force includes and/or refers to a phenomenon and/or a force exerted on a particle when subjected to a non-uniform electric field. The DEP actuator 108 may include a DEP electrode that is used to apply a non-uniform electric field on a particle, such as a cell or clump of cells, passing by the DEP actuator 108. The DEP force is applied on the particle when the particle passes through the non-uniform electric field. A non-uniform electric field, as used herein, includes and/or refers to an electric field with a magnitude and/or direction of electric intensity which differs or is not the same at all points of the electric field. The strength of the DEP force applied is dependent on the fluid the particle is in, electrical properties of the particle, particle shape and size, and the frequency of the non-uniform electric field. The non-uniform electric field may polarize the particle, with the pole experiencing the DEP force along the field lines. As the electric field is non-uniform, the pole experiencing the greater electric force may dominate over the other, causing the particle to change altitude positions within the microfluidic channel 102, as further described herein.

The microfluidic device 100 further includes a fluid ejection device 110 fluidically coupled to the microfluidic device 100. The fluid ejection device 110 may actuate to cause flow of the fluid from the reservoir 103 and along the microfluidic channel 102, such that a plurality of particles within the fluid pass over the first sensor 104, the second sensor 106, and the DEP actuator 108. The fluid ejection device 110 may further be actuated to eject a volume of the fluid from the microfluidic device 100. The fluid ejection device 110 includes an ejection chamber 113 with a fluid actuator 109 and a nozzle 107 to eject fluid from the microfluidic device 100. The ejection chamber 113 is fluidically coupled to the nozzle 107, and with the fluid actuator 109 disposed in the ejection chamber 113. The nozzle 107 may include an orifice used for ejecting fluid from the ejection chamber 113. A fluid actuator, as used herein, includes and/or refers to circuitry and/or a physical structure that causes movement of fluid. Example fluid actuators include an integrated inertial pump, a thermal inkjet (TIJ) resistor, a piezoelectric device, a magnetostrictive element, an electrode, an ultrasound source, mechanical/impact driven membrane actuators, magneto-restrictive drive actuators, and other suitable components.

For example, the fluid ejection device 110 may include a drop-on-demand thermal bubble system including a TIJ ejector. The TIJ ejector may implement a thermal resistor in the ejection chamber 113 and create bubbles that force fluid drops out of the nozzle 107. In some examples, the fluid may be ejected from the microfluidic device 100 by the fluid ejection device 110 that includes a drop-on-demand piezoelectric inkjet system including a piezoelectric inkjet (PIJ) ejector that implements a piezoelectric material actuator as an ejection element to generate pressure pulses that force fluid drops out of the nozzle 107. Examples are not so limited and additional and/or different types of fluid ejection device 110 may be used to eject fluid from the ejection chamber 113. Similarly, different and/or additional components may be coupled to the microfluidic device 100 to eject fluid therefrom, such as a fluid dispensing device and other components.

FIG. 1B illustrates a side view of the microfluidic device 100 illustrated by FIG. 1B. As shown, a first particle 105 may flow from the reservoir (e.g., reservoir 103 illustrated by FIG. 1A) into the interrogation region 111 of the microfluidic channel 102 that includes the first and second sensors 104, 106 and the DEP actuator 108. The fluid flow may be at a constant velocity, in some examples, such that changes in velocity between different particles may yield further information on particle properties.

In various examples, the first sensor 104 is to provide a first electric field within the microfluidic channel 102, and in response, obtain a first sensor signal associated with a first particle 105 within the fluid as the first particle 105 passes by the first sensor 104 in the microfluidic channel 102. The DEP actuator 108 is to provide a non-uniform electric field within the microfluidic channel 102 as the first particle 105 passes by the DEP actuator 108 within the microfluidic channel 102. The first particle 105 may be drawn toward or away from the DEP actuator 108 in response to the non-uniform electric field depending on particle properties. The second sensor 106 is to provide a second electric field within the microfluidic channel 102, and in response, obtain a second sensor signal associated with the first particle 105 within the fluid as the first particle 105 passes by the second sensor 106 in the microfluidic channel 102. The first electric field and second electric field may include uniform electric fields which are used to obtain sensor signals indicative of impedance-based measures and/or capacitance-based measures.

The sensor signals obtained before and after the DEP force is applied may be used to determine at least one particle property. The particle property includes and/or refers to an electrical property and/or mechanical property exhibited by a particle. Example electrical properties include dielectric properties, such as a positive or negative dielectrophoresis at a particular frequency and a cross-over frequency, among others. Example mechanical properties include a size, velocity, and flexibility of a cell or other particle, such as a clump of cells.

For example, the interrogation region 111 is used to obtain the sensor signals which are indicative of an altitude of the first particle 105 before and after the DEP force is applied thereto. Changes in altitude may provide an indication of particle properties, such as a type of particle and/or a size of the particle. In some examples, the difference in altitude position of the first particle 105 determined using the first sensor signal and the second sensor signal may yield information on the first particle 105 experiencing positive or negative dielectrophoresis, which may be used to determine a particle type, such as a type of cell. The altitude position may be determined based on peaks of the first sensor signal and the second sensor signal. Particle type includes and/or refers to a classification of a particle and/or particle feature, such as whether particle is a cell or other cellular or molecular material, a clump of cells, a particular type of cell, and/or the cell is alive or dead. Example particle types include cells, clumps of cells, debris, portions of cellular or molecular material, such as cell membranes, liposomes, proteins, and nucleic acid sequences. Example cell types include an alive cell, a dead cell, and different classes of cells, such as blood cells (e.g., red blood cells, white blood cells, platelet), stem cells, sex cells (e.g., sperm cells, egg cells), fat cells, nerve cells, muscle cells, and bone cells. Specific cell or particle types include an antibody, an enzyme, T-cell, B-cell, hormones, blood factors, viruses, dendritic cells, macrophages, among other types of cells. In some examples, the particle type may include identification of a population or number of cells, such as a clump of many cells.

In some examples, the dielectric properties of the particles may be assessed to determine the particle type. For example, the membrane of cells consist of a lipid bilayer which is insulating with a conductivity of about 10\hat{ }-7 S/m. The conductivity of the cytoplasm, which is an interior part of the cell, may be as high as 1 S/m. Upon cell death, the cell membrane becomes permeable, resulting in increased conductivity of dead cells compared to alive cells by a factor of 10\hat{ }4. The change in conductivity may cause different responses to the non-uniform electric field, such as a dead cell being drawn closer to the DEP actuator 108 than an alive cell of the same cell type.

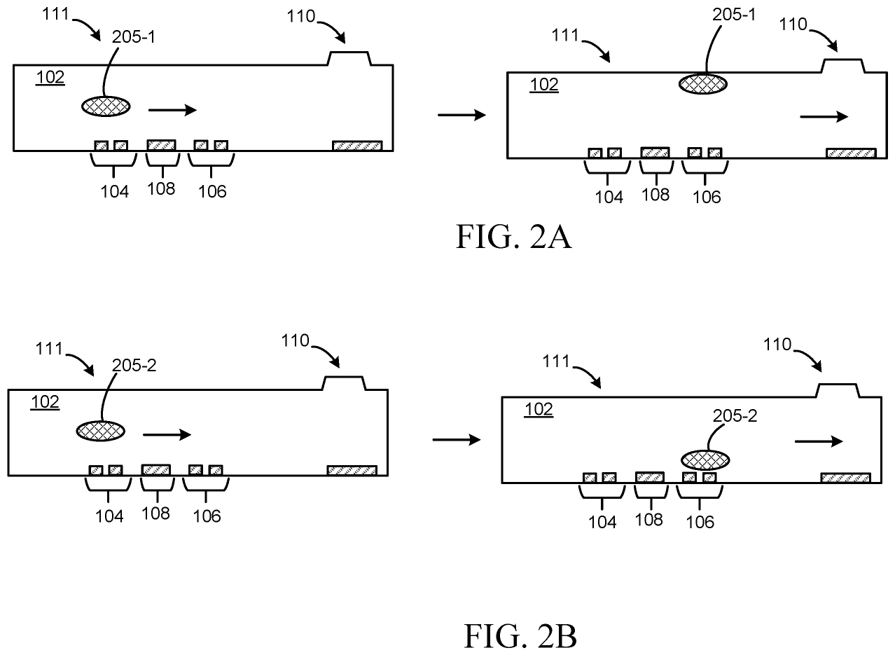

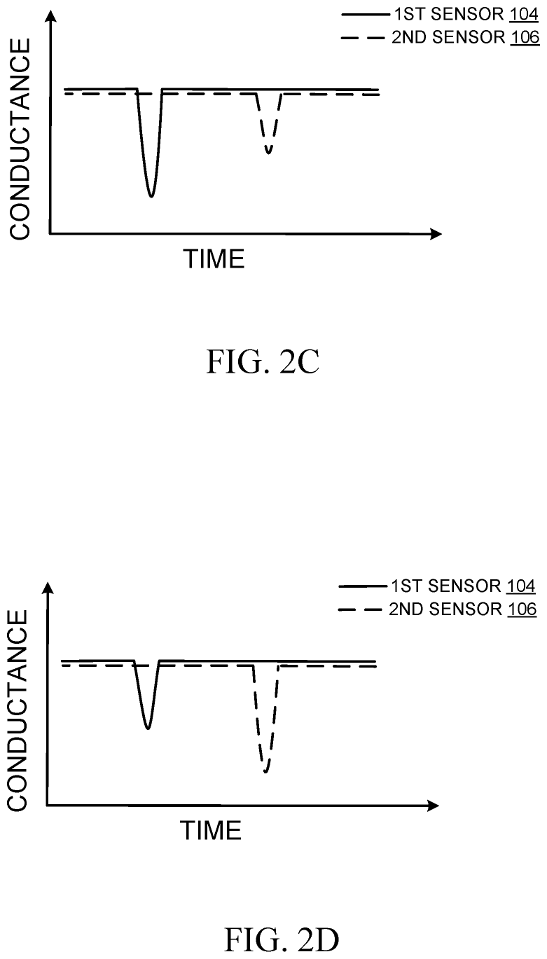

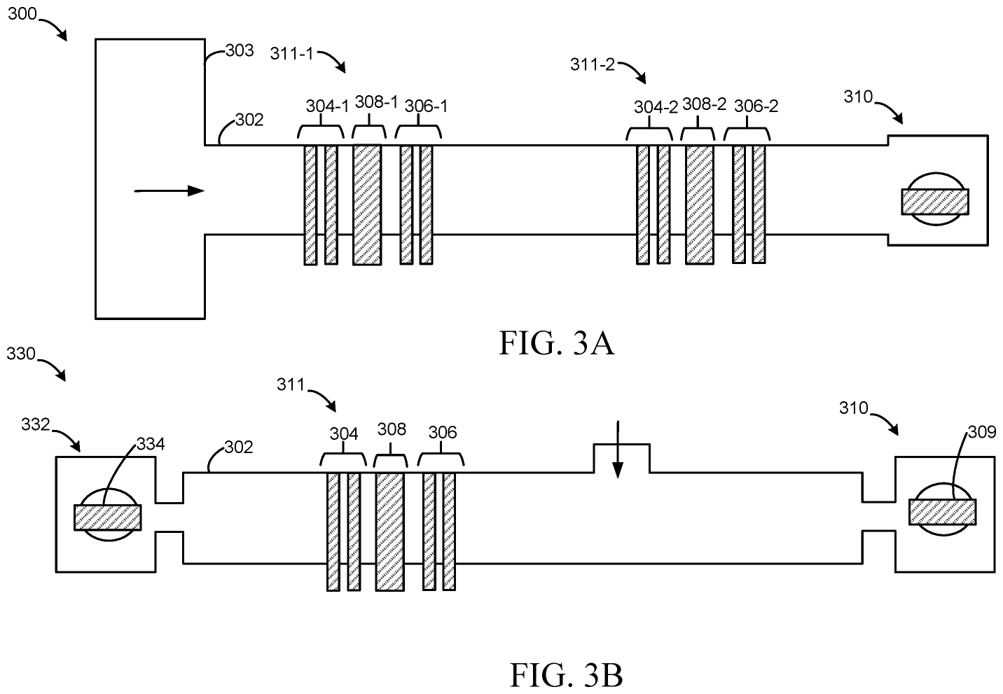

In some examples, other types of particle properties may be determined based on the signal shapes of the sensor signals, timing between peaks of the sensor signals, and other information, as further illustrated and described by FIGS. 2C-2D, such as mechanical properties. For example, the timing between the peaks may be indicative of the velocity of the first particle 105. As the flow of fluid is driven at a constant velocity, the velocity may be indicative of the resistance the first particle 105 experiences while traversing through the microfluidic channel 102, which may be indicative of the size of the first particle 105. Further, the shape of the sensor signals may be indicative the particle size and/or flexibility.

As an example, a first type of particle may be expected to take a certain amount of time between the first sensor 104 and the second sensor 106, depending on the size of the particle. A clump of cells and/or other types of particles may traverse the path longer and therefore may be classified as a different type of particle, such as being classified as waste or a clump of cells. The bound may be soft, with clumps or particles taking longer time than a threshold being assigned a lower probability of being the particle of interest.

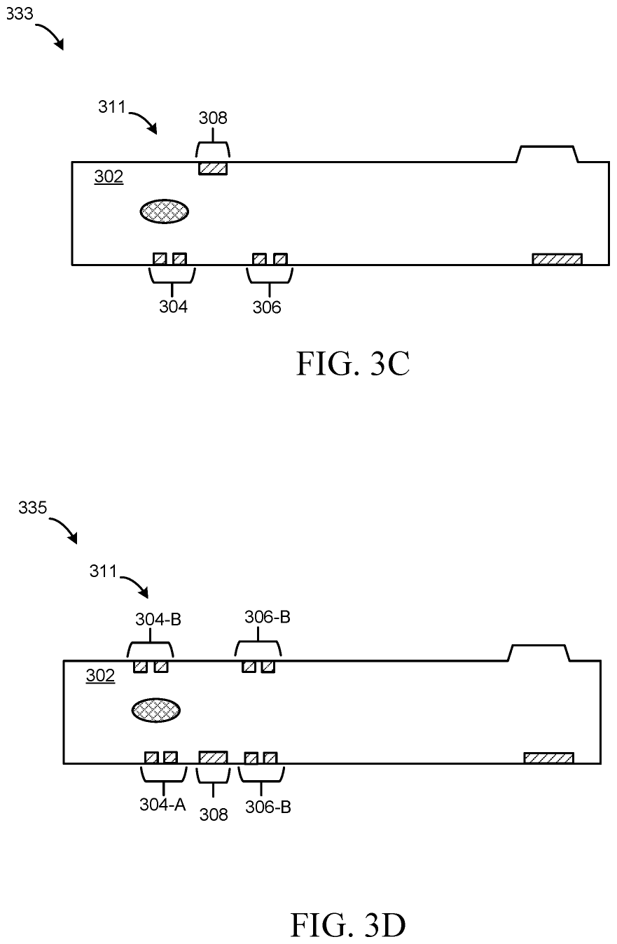

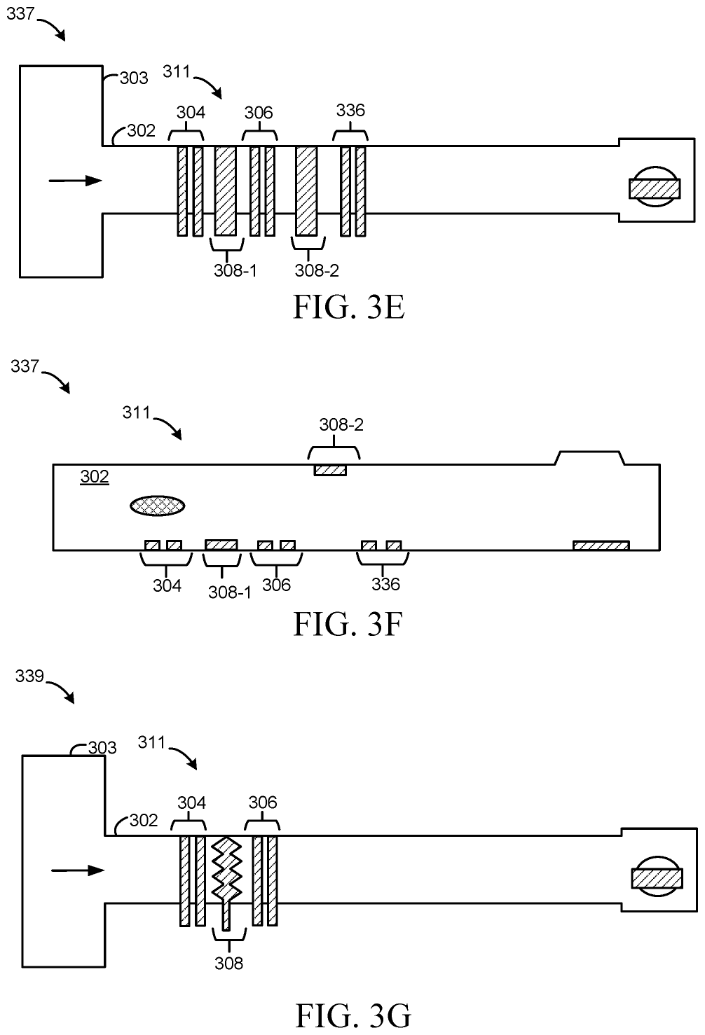

In various examples, the microfluidic device 100 of FIGS. 1A-1B includes additional components, such as additional microfluidic channels, interrogation regions, sensors, DEP actuators, fluid actuators, chambers, actuators, fluid reservoirs, and other components. For example, and as further illustrated and described by FIG. 3A, the interrogation region 111 may include a first interrogation region and the microfluidic device 100 may further include a second interrogation region between the (first) interrogation region 111 and the fluid ejection device 110. The second interrogation region may include a third sensor, a fourth sensor, and a second DEP actuator disposed within the microfluidic channel, with the second DEP actuator being disposed between the third sensor and the fourth sensor. In some examples, as further illustrated by FIG. 3B, the microfluidic device 100 may further include a fluid actuator fluidically coupled to the microfluidic channel 102, where the first sensor 104, the second sensor 106, and the DEP actuator 108 are disposed between the fluid ejection device 110 and the fluid actuator within the microfluidic channel 102. In some examples, as illustrated by FIG. 3E, the microfluidic device 100 may include a second DEP actuator and a third sensor disposed within the microfluidic channel 102, where the second DEP actuator is disposed between the second sensor 106 and the third sensor. The DEP actuator 108 and the second DEP actuator may be disposed on the same wall or a different wall of the microfluidic channel 102.

FIGS. 2A-2D illustrate example operations of a microfluidic device with a DEP actuator, consistent with the present disclosure. The microfluidic device of FIGS. 2A-2B may include an implementation of and/or include substantially the same features and components as the microfluidic device 100 of FIG. 1A, with the common features and components similarly labeled and not repeated.

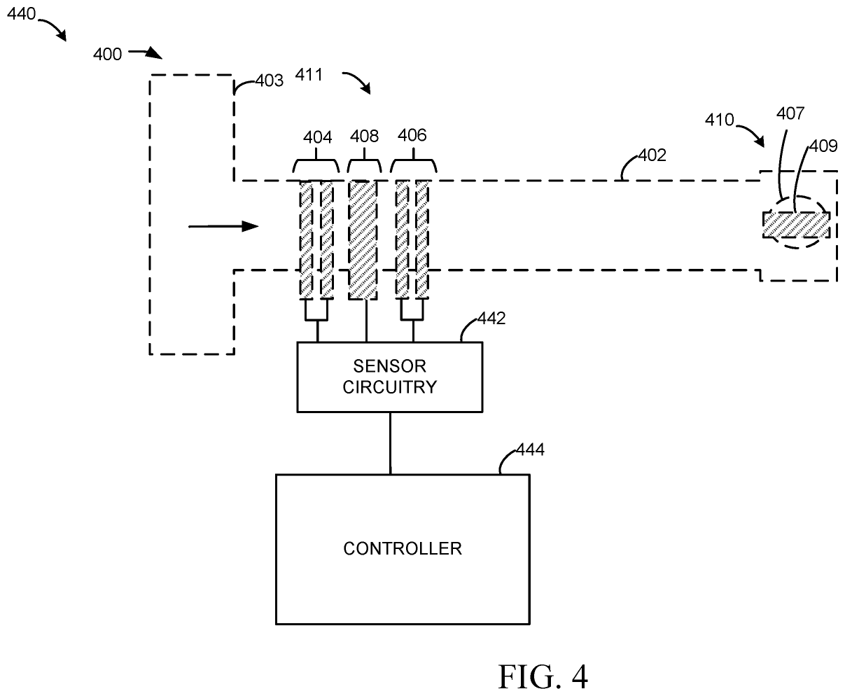

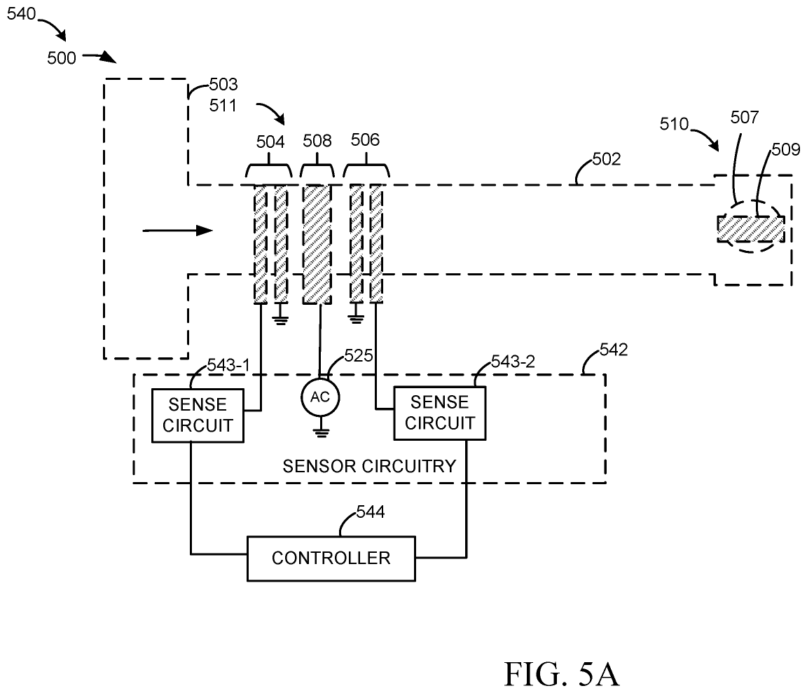

In some examples, a fluid ejection device 110 may be used to provide a constant fluid flow through the microfluidic channel 102 and to eject a volume of the fluid to a plurality of regions of the substrate. A coupled controller, as further illustrated by FIG. 4, may control firing of the fluid ejection device 110 by sending electrical signals to the fluid ejection device 110 via electrical connects, and may determine particle properties of a particle within the fluid based on sensor signals received from the first sensor 104 and the second sensor 106. The controller may classify and record, in a dispense map, an indication of particle properties of particles dispensed into each of a plurality of regions of the substrate, as further described herein.

FIG. 2A illustrates a first particle 205-1 flowing through the interrogation region 111 of the microfluidic device. In some examples, the fluid ejection device 110 is actuated to draw fluid into the interrogation region 111. As shown by the left side of FIG. 2A, as the first particle 205-1 flows over the first sensor 104, a first sensor signal is obtained using the first sensor 104. The first particle 205-1 is at a first altitude position within the microfluidic channel 102, which may be random. The closer the first particle 205-1 is to the first sensor 104, the greater the impedance-based measure (or other signal measure). The first sensor 104 may obtain an impedance-based measure as a sensor signal that is dependent on the altitude position of the first particle 205-1, e.g., how close the first particle 205-1 is to the first sensor 104. As the first particle 205-1 flows over the DEP actuator 108, the first particle 205-1 is either deflected away from or toward the DEP actuator 108, depending on whether the first particle 205-1 experiences a negative or positive dielectrophoresis at a frequency of operation of the DEP actuator 108.

The right side of FIG. 2A shows the first particle 205-1 after experiencing the DEP force by the DEP actuator 108. In the example, the first particle 205-1 experiences a negative dielectrophoresis (nDEP) and is pushed away from the DEP actuator 108. As the first particle 205-1 flows over the second sensor 106, a second sensor signal is obtained using the second sensor 106. For example, the second sensor 106 may obtain an impedance-based measure as a sensor signal that is dependent on the altitude position of the first particle 205-1 as the first particle 205-1 passes over the second sensor 106. In the example, the first particle 205-1 is at a second altitude position within the microfluidic channel 102 that is greater than the first altitude position illustrated by the left side of FIG. 2A.

FIG. 2C illustrates resulting first and second sensor signals obtained by the first and second sensors 104, 106 as associated with the first particle 205-1. As shown, the first sensor signal has a higher peak than the second sensor signal due to the change in altitude position. The difference between the peaks may yield information on particle properties of the first particle 205-1.

FIG. 2B illustrates a second particle 205-2 flowing through the interrogation region 111 of the microfluidic device. As shown by the left side of FIG. 2B, as the second particle 205-2 flows over the first sensor 104, a first sensor signal is obtained using the first sensor 104. As the second particle 205-2 flows over the DEP actuator 108, the second particle 205-2 is either deflected away from or toward the DEP actuator 108, as previously described by FIG. 2A.

The right side of FIG. 2B shows the second particle 205-2 after experiencing the DEP force by the DEP actuator 108. In the example, the second particle 205-2 experiences a positive dielectrophoresis (pDEP) and is pulled toward the DEP actuator 108. As the second particle 205-2 flows over the second sensor 106, a second sensor signal is obtained using the second sensor 106. In the example, the second particle 205-2 is at a second altitude position within the microfluidic channel 102 that is less than the first altitude position illustrated by the left side of FIG. 2B.

FIG. 2D illustrates resulting first and second sensor signals obtained by the first and second sensors 104, 106 as associated with the second particle 205-2. As shown, the first sensor signal has a lower peak than the second sensor signal due to the change in altitude position. The difference between the peaks may yield information on particle properties of the second particle 205-2.

FIGS. 20-2D illustrate example first sensor signals and second sensor signals which provide an indication of a change in impedance and/or capacitance associated with a particular particle. The change in impedance and/or capacitance indicates the change in altitude position of the particle in response to the DEP force, and indicates whether the particle experiences the pDEP or nDEP. Different types of particles may experience pDEP or nDEP at particular frequencies, such as dead cells verses alive cells or different size or types of particles, among other differences. As the flow of fluid is kept constant, the timing difference between peaks of a first sensor signal and a second sensor signal associated with a particle may provide information on the size of the particle. Furthermore, the shape of the sensor signals, such as the width of the peaks, may provide information on particle size and/or flexibility.

In some examples, the expected dielectric and/or mechanical properties, velocity, and/or signal shapes for different types of particles may be known and/or stored in data. In some examples, the microfluidic device may have another fluid flown there through, with known types of particles, and which is used to determine expected dielectric properties (e.g., pDEP or nDEP and/or cross-over frequency), velocity, and/or signal shapes for different types of particles through a calibration process. The dielectric and/or mechanical properties, velocity, and/or signal shapes for the different particle types may be determined and stored in a data map.

FIGS. 3A-3G illustrate further example microfluidic devices with DEP actuators, consistent with the present disclosure. The microfluidic devices of FIGS. 3A-3G include substantially the same features and components as the microfluidic device of FIGS. 1A-1B, with some variations as further described herein and are numbered accordingly. For instance, each of the microfluidic devices 300, 330, 333, 335, 337, 339 of FIGS. 3A-3G include a microfluidic channel 302 fluidically coupled to a reservoir 303 and having an interrogation region 311 (or 311-1) including a first sensor 304 and a second sensor 306 (or 304-1, 306-1 or 304-A, 304-B and 306-A, 306-B) and a DEP actuator 308 (or 308-1), and a fluid ejection device 310 fluidically coupled to the microfluidic channel 302. As previously described, the microfluidic device 100 of FIGS. 1A-1B may include a variety of variations, at least some of which are illustrated by FIGS. 3A-3G.

FIG. 3A illustrates an example microfluidic device 300 which includes a microfluidic channel 302 including a first interrogation region 311-1 and a second interrogation region 311-2. The first interrogation region 311-1 includes a first sensor 304-1, a second sensor 306-1, and a first DEP actuator 308-1, as previously described. The second interrogation region 311-2 includes a third sensor 304-2 and a fourth sensor 306-2 disposed within the microfluidic channel 302, and a second DEP actuator 308-2 disposed within the microfluidic channel 302 between the third sensor 304-2 and the fourth sensor 306-2. The first interrogation region 311-1 may be upstream from the second interrogation region 311-2, as shown by FIG. 3A.

In some examples, the first DEP actuator 308-1 and the second DEP actuator 308-2 may operate at fixed frequencies. For example, the first DEP actuator 308-1 may operate at a first frequency and the second DEP actuator 308-2 may operate a second frequency that is different than the first frequency. The fixed frequencies may be set based on an application, such as particle types and/or sample type. As particles pass through each of the first interrogation region 311-1 and the second interrogation region 311-2, different DEP forces are experienced and the resulting sensor signals are obtained.

Although the example illustrates two interrogation regions 311-1, 311-2, examples are not so limited and may include additional interrogation regions in the microfluidic channel 302. In some examples, the plurality of interrogation regions may be used to measure when and/or at what frequency a particle experiences a shift from a pDEP to a nDEP (or vice versa), herein generally referred to as the cross-over frequency. For example, the cross-over frequency may be interpolated from the sensor signals. By fixing the frequencies that the first and second DEP actuators 308-1, 308-2 operate at, dedicated signal generation circuitry may be used. Further, several particles may traverse the plurality of interrogation regions at the same time, with each particle being within a different interrogation region at the particular time. The cross-over frequency may be used to determine the type of particle and/or with better confidence, as different particle types (e.g., T-cells, B-cells, red blood cells) may have defined cross-over frequencies.

In some examples, the different interrogation regions 311-1, 311-2 may operate at adaptive frequencies, which may be adaptive based on particle responses to the DEP force(s). For example, the operating frequency of the second interrogation region 311-2 may be adjusted based on a particle response to the first interrogation region 311-1. As a particular example, the first DEP actuator 308-1 may operate at a frequency below the lowest expected cross-over frequency, and the second DEP actuator 308-2 may operate at a frequency above the highest expected cross-over frequency. In some examples, although not illustrated, the microfluidic device 300 may include additional downstream interrogation regions. For example, a third interrogation region may be disposed downstream from the second interrogation region 311-2, with a third DEP actuator that operates at a frequency between the frequencies of the first and second DEP actuators 308-1, 308-2, such as an average of the two frequencies. In some examples, a fourth (or more) interrogation region is downstream from the third interrogation region, which has a frequency selected based on sensor signals from the third interrogation region. In some examples, the frequency may be selected based on a bisection technique, such as a binary search method, among other techniques, such as Newton Ralphson method, secant method, regula falsi method, and/or the Steffenson's method. The cross-over frequency may thereby be identified by setting or adjusting the frequencies of the first and/or second DEP actuators 308-1, 308-2.

By using the multiple interrogation regions and/or adaptive frequencies, the cross-over frequency may be determined and used to better classify the particle type and to provide a distribution of the particle population cross-over frequency for a sample fluid. Estimating the cross-over frequency of a particle population from a first sample, may be used to set a search range for a second sample, and allowing for the adaptive frequency operation to converge to a more accurate cross-over frequency faster and using less interrogations and/or resulting in higher precision in the cross-over frequency determination.

FIG. 3B illustrates an example microfluidic device 330 which includes a microfluidic channel 302 including an interrogation region 311, a fluid ejection device 310 including a (first) fluid actuator 309, and a (second) fluid actuator 334 fluidically coupled to the microfluidic channel 302. The (second) fluid actuator 334 may form part of a second fluid ejection device 332 and/or may include a fluid actuator 334 that forms part of the microfluidic channel 302. The fluid actuator 334 and fluid ejection device 310 may be used to provide higher resolution determination of particle properties by moving a particle over the interrogation region 311 multiple times and obtaining different sensor signals by operating the DEP actuator 308 at different frequencies each time.

In some examples, the interrogation region 311 may be disposed between the fluid ejection device 310 and the fluid actuator 334 within the microfluidic channel 302. For example, the reservoir containing the fluid may be coupled to an inlet of the microfluidic device 330, as illustrated by the arrow of FIG. 3B. The fluid actuator 334 may be actuated to pull the particle into the microfluidic channel 302 and across the interrogation region 311. The fluid actuator 309 of the fluid ejection device 310 may be actuated to pull the particle over the interrogation region 311 a second time. In some examples, the process may be repeated a number of times, with the frequency of the DEP actuator 308 being adjusted each time. After the process is complete, the fluid actuator 309 of the fluid ejection device 310 may be further actuated to dispense the particle to a region of a substrate.

In some examples, based on the results of the multiple measurements, a decision may be made to dispense the particle to a first location or a second location, such as a first region or second region of a substrate or a first region of the substrate and a waste reservoir, using either the fluid ejection device 310 or the second fluid ejection device 332. For example, a coupled controller may determine the location to dispense the particle to based on a determined particle property, and selectively actuate one of the fluid ejection device 310 or the second fluid ejection device 332 to eject the particle to the location. Particles may be dispensed to the different locations with or without moving a stage, as further illustrated herein.

As with FIG. 3A, the different frequencies of the DEP actuator 308 may be fixed frequencies or may be adaptive. For example, the microfluidic channel 302 may include a plurality of interrogation regions with different DEP actuators and each DEP actuator may be at a fixed frequency, as described by FIG. 3A. In other examples or in addition, the microfluidic channel 302 may include the interrogation region 311 and the DEP actuator 308 may be transitioned through a set of fixed frequencies. In other examples, the microfluidic channel 302 may include the interrogation region 311 and the DEP actuator 308 is adaptively adjusted after measuring at the lowest expected frequency, the highest expected frequency, and the average between the lowest and highest expected frequencies, with a particle drawn back and forth through the interrogation region 311.

FIG. 3C illustrates an example microfluidic device 333 with a microfluidic channel 302 and an interrogation region 311 including the first sensor 304 and the second sensor 306 on a first wall, e.g., the floor, of the microfluidic channel 302, and the DEP actuator 308 on a second wall, e.g., the ceiling, of the microfluidic channel 302. The second wall is opposite the first wall.

FIG. 3D illustrates an example microfluidic device 335 including with a microfluidic channel 302 and an interrogation region 311 including the first sensor and the second sensor on a first wall, e.g., the floor, and a second wall, e.g., the ceiling, of the microfluidic channel 302, and the DEP actuator 308 on the first wall of the microfluidic channel 302. The second wall is opposite the first wall. In such examples, the first sensor includes a first pair of sensing electrodes 304-A on the first wall and a second pair of sensing electrodes 304-B on the second wall. The second sensor includes a first pair of sensing electrodes 306-A on the first wall and a second pair of sensing electrodes 306-B on the second wall. Placing sensing electrodes 304-A, 304-B, 306-A, 306-B on both the first and second walls may be used to measure the particle altitude position within the microfluidic channel 302 with greater certainty than with one pair of sensing electrodes per sensor.

FIGS. 3E-3F illustrate an example microfluidic device 337 which includes a microfluidic channel 302 with the interrogation region 311 that includes the first sensor 304, the first DEP actuator 308-1, the second sensor 306, a second DEP actuator 308-2, and a third sensor 336. The second DEP actuator 308-2 and third sensor 336 are disposed within the microfluidic channel 302, with the second DEP actuator 308-2 being disposed between the second sensor 306 and the third sensor 336.

As shown by the side view of FIG. 3F, in some examples, the first DEP actuator 308-1 is disposed on a first wall of the microfluidic channel 302 and the second DEP actuator 308-2 is disposed on a second wall of the microfluidic channel 302 that is opposite the first. In such examples, the first and second DEP actuators 308-1, 308-2 may operate at the same frequency, and which may result in a particle being pushed (or pulled) by the first DEP actuator 308-1 and pulled (or pushed) by the second DEP actuator 308-2 to provide greater confidence on the nature of motion caused by the DEP force. However, examples are not so limited, and the first and second DEP actuators 308-1, 308-2 may be located on the same wall, and/or may operate at different frequencies.

FIG. 3G illustrates an example microfluidic device 339 which includes a microfluidic channel 302 with the interrogation region 311 that includes the first sensor 304, the second sensor 306, and a DEP actuator 308 that includes a saw-toothed or curved electrode. The saw-toothed electrode may increase a surface area of the DEP electrode and result in increased local gradient of the non-uniform electric field.

FIG. 4 illustrates an example apparatus including sensor circuitry and a controller, consistent with the present disclosure.

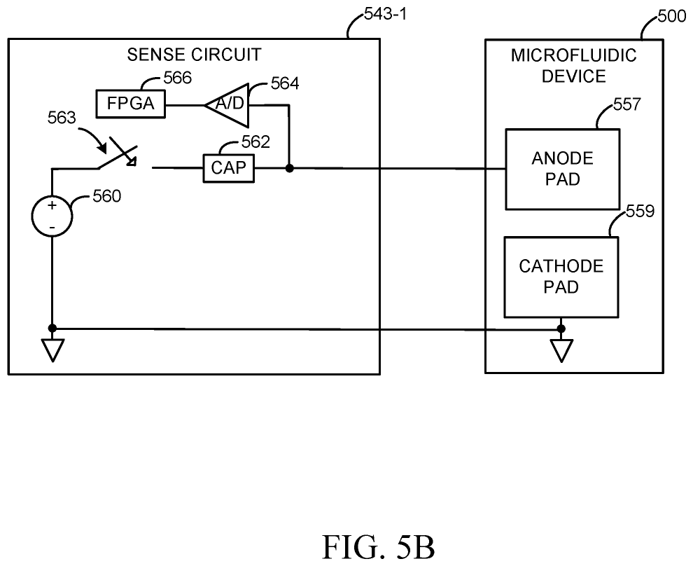

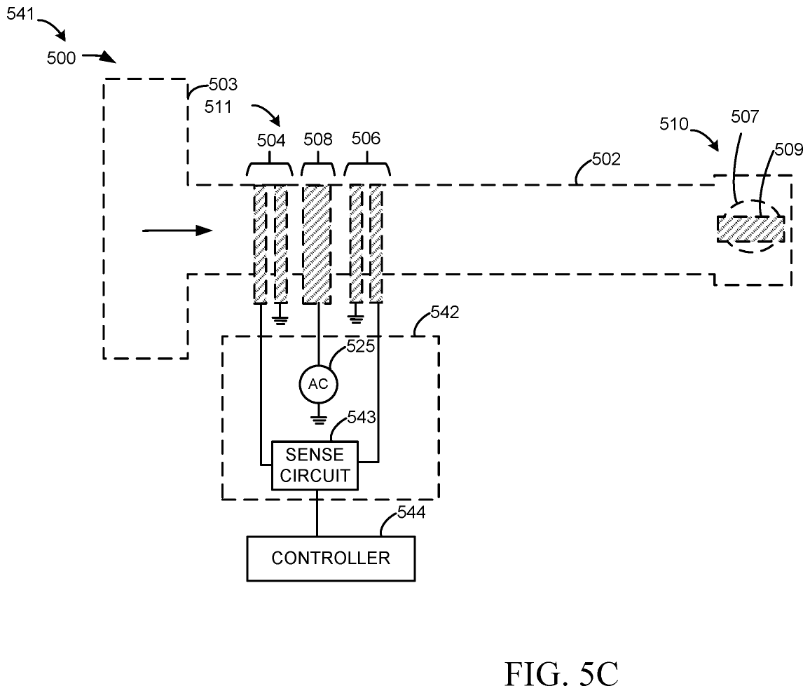

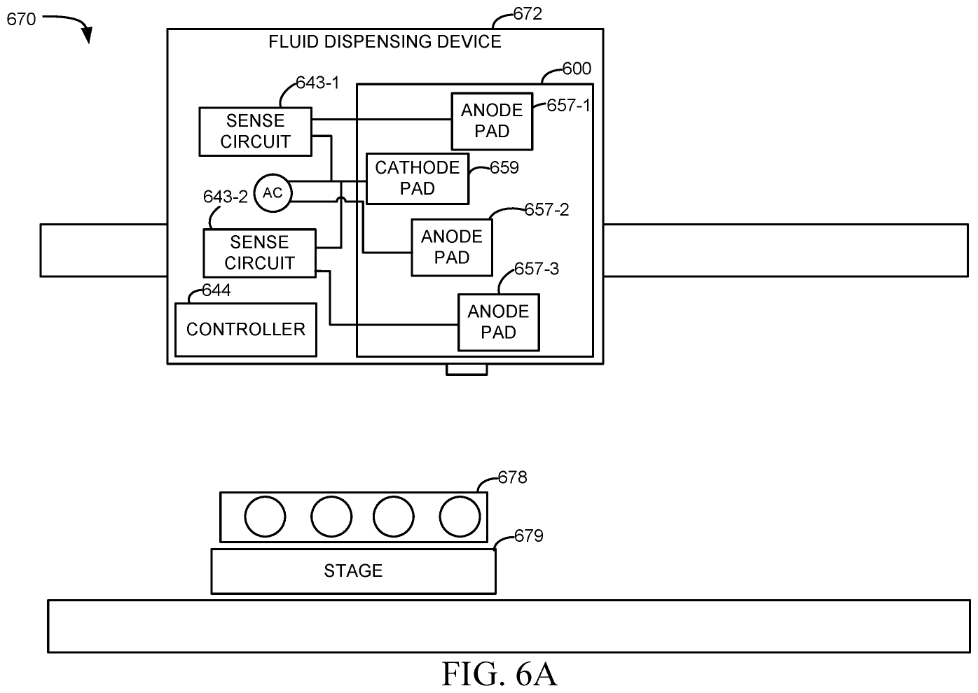

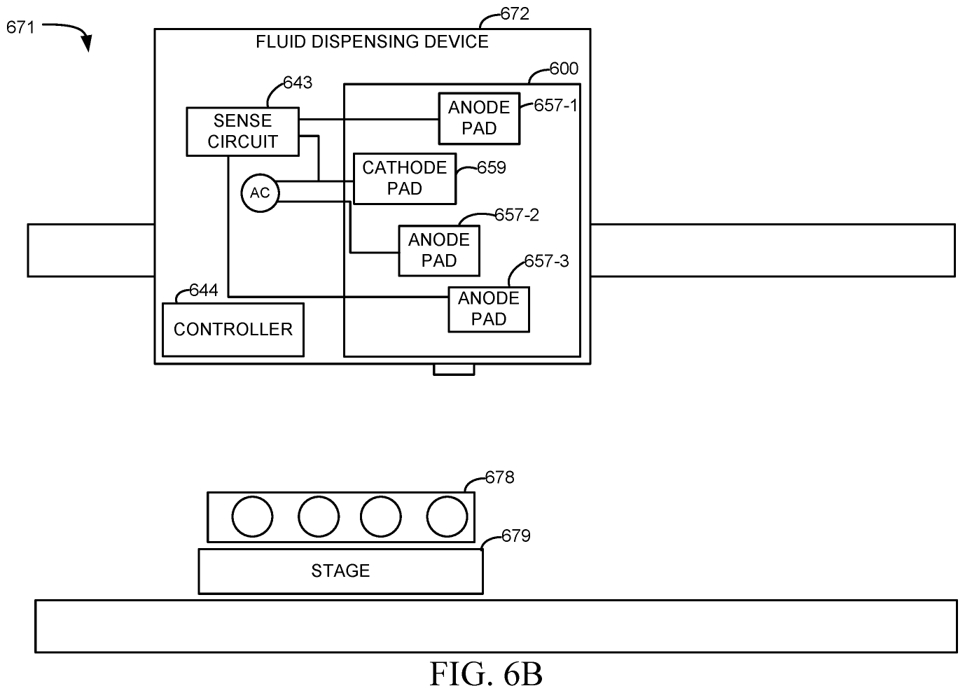

The apparatus 440 of FIG. 4 (as well as FIGS. 5A-5B and 6) may include or form part of a fluid dispensing device. A fluid dispensing device may be an ink-jet based dispensing device that may dispense picoliters or nanoliters of fluid into specific locations on a substrate. In various examples, a fluid dispensing device may use a microfluidic device 400 for dispensing fluid. The microfluidic device 400 may operate similar to a printhead. The fluid dispensing device may include a substrate transport assembly to move the substrate and circuitry including the sensor circuitry 442 and controller 444. The fluid dispensing device may include additionally non-illustrated components, such as a mounting assembly and a power supply that provides power to the various electrical components of the fluid dispensing device and the microfluidic device 400 mounted therein.

As shown by FIG. 4, an example apparatus 440 includes sensor circuitry 442 and a controller 444. The sensor circuitry 442 may couple to an interrogation region 411 of a microfluidic channel 402 of a microfluidic device 400 and obtain sensor signals using a first sensor 404 and a second sensor 406. The microfluidic device 400 may include an implementation of and/or include substantially the same features and components as any of the microfluidic devices 100, 300, 330, 333, 335, 337, 339 of FIGS. 1A-1B and FIGS. 3A-3G, such as including a microfluidic channel 402 coupled to a reservoir 403, an interrogation region 411, and a fluid ejection device 410 including a nozzle 407 and fluid actuator 409. As shown, the interrogation region 411 includes the first sensor 404, the second sensor 406, and the DEP actuator 408 between the first sensor 404 and the second sensor 406.

In some examples, the apparatus 440 includes the microfluidic device 400 including the microfluidic channel 402 and the fluid ejection device 410. The sensor circuitry 442 is coupled to the first sensor 404 and the second sensor 406 to obtain sensor signals. As further described below, the sensor signals may include a first sensor signal associated with a particle as the particle passes by the first sensor 404 in the microfluidic channel 402 and a second sensor signal associated with the particle as the particle passes by the second sensor 406 in the microfluidic channel 402. As further illustrated by FIG. 5A, the sensor circuitry 442 may include a first sense circuit and a second sense circuit respectively coupled to the first sensor 404 and the second sensor 406.

The controller 444 is coupled to the sensor circuitry 442 to cause flow of fluid including a particle through the interrogation region 411 via actuation of the fluid ejection device 410 coupled to the microfluidic channel 402, and apply electric fields within the microfluidic channel 402 via the first sensor 404, the second sensor 406, and the DEP actuator 408, the electric fields including a non-uniform electric field applied via the DEP actuator 408. The fluid ejection device 410 may be actuated by the controller 444 sending or transmitting an electrical signal to the fluid actuator 409 of the fluid ejection device 410 to cause the fluid actuator 409 to actuate and which causes the fluid to flow, as previously described. The controller 444 may apply the electric fields by transmitting electrical signals to the sensor circuitry 442, which causes the application of the electric fields within the microfluidic channel 402 via the first sensor 404, the DEP actuator 408, and the second sensor 406.

The controller 444 determines a particle property of the particle using the sensor signals received from the sensor circuitry 442 responsive to the flow of the particle through the interrogation region 411. For example, the controller 444 and the sensor circuitry 442 apply the electric field including a first electric field, the non-uniform electric field applied via the DEP actuator 408, and a second electric field. The first electric field is applied via the first sensor 404 and used to measure the first sensor signal in response. The second electric field is applied via the second sensor 406 and used to measure the second sensor signal in response, wherein the controller 444 is to determine the particle property based on a comparison between the first sensor signal and the second sensor signal responsive to the application of the non-uniform electric field.

The controller 444 further causes the fluid ejection device 410 to eject the particle from the microfluidic device 400 to a select region of a substrate based on the determined particle property. The region may be selected based on the particle property, such as dispensing dead cells or clumps of cells and/or debris to a waste region, single cells or other particles of a particular type to a select region or a group of regions, among other variations. The particle may be ejected by ejecting a volume of the fluid containing the particle via an ejection chamber with the fluid actuator 409 and the coupled nozzle 407. As previously described, the fluid actuator 409 may include a resistor, and the controller 444 may actuate the resistor of the fluid ejection device 410 to cause the ejection of the volume of the fluid by transmitting or applying an electrical signal to the resistor.

In some examples, the fluid includes a plurality of particles, the plurality of particles including the particle. The controller 444 may determine particle properties of the plurality of particles, cause the fluid ejection device 410 to eject each of the plurality of particles from the microfluidic device 400 to select regions of the substrate based on the determined particle properties, and store a dispense map indicative of the select regions of the substrate that the plurality of particles are ejected to and as associated with the respective particle properties of the plurality of particles.

A dispense map, as used herein, includes and/or refers to data identifying particle properties of particle(s) within regions or classifying particles within the regions, e.g., wells, of a substrate. For example, the region may be classified as including a particular type of particle (e.g., cell or not, dead or alive) and/or a target number of particles or a single particle. The dispense map may identify regions of the substrate with target particle populations. The change in peak heights, peak widths, and time between the first and second sensor signals may be used to identify the particle properties, such as the type of particle, the size of the particle, identify clusters of particles, debris, and/or signal noise. Based on the determined particle properties, the dispense map may be generated by indicating which region of the substrate includes target particles, a target number of particles, and/or waste. In some examples, the map may be generated in real time and/or on-the-fly while the apparatus 440 is continuing to dispense fluid into further regions of the substrate. As used herein, real time includes and/or refers to processing of signals or other data within a threshold amount of time, e.g., seconds or milliseconds. On-the-fly, as used herein, includes and/or refers to processing that occurs while the apparatus 440 is in motion and/or another process is in progress.

A target particle population, as used herein, includes and/or refers to a defined number of particles, or n-particles, of a particular type to be dispensed into a region of a substrate. A target particle population may include a single particle or multiple particles. In some examples, the target particle population includes a single cell and/or a specific type of cell which may be identified by the particle properties detected. However, examples are not so limited. In various examples, different regions of the substrate may have different target particle populations. The region includes and/or refers to a particular location of a substrate to which a particle or a target particle population is to be dispensed. The region may be a particular well on a microwell plate or other types of substrates.

In some examples, the controller 444 may set the frequency of the DEP actuator 408. For example, the frequency may be set as a fixed frequency for different applications and/or may be adjusted for determining a cross-over frequency of a particle and/or distribution of particles of a particle population, as previously described. In various examples, the frequency of the DEP actuator 408 (or any of the other DEP actuators described herein) may be set to a range between about 10 kilohertz (kHz) and 1 megahertz (MHz). In some examples, such as with mammalian cells, cross-over frequencies for particles may be in a range between about 50 kHz and about 300 kHz. However, examples are not so limited and other frequency ranges may be used.

In some examples, the controller 444 may be used in a calibration process to determine different expected particle properties of a plurality of different particles. The different particle properties may include different electrical and mechanical properties and/or responses of the cells and other particles to the electric fields and while flown through the microfluidic channel 402 of the microfluidic device 400.

The controller 444 may include a processor and memory. Memory may include a computer-readable storage medium storing a set of instructions. Computer-readable storage medium may include Read-Only Memory (ROM), Random-Access Memory (RAM), Electrically Erasable Programmable Read-Only Memory (EEPROM), flash memory, a solid state drive, physical fuses and e-fuses, and/or discrete data register sets. In some examples, computer-readable storage medium may be a non-transitory storage medium, where the term “non-transitory” does not encompass transitory propagating signals.

The processor may be a central processing unit (CPU), a semiconductor-based microprocessor, a graphics processing unit (GPU), a microcontroller, special purpose logic hardware controlled by microcode or other hardware devices suitable for retrieval and execution of instructions stored in the non-transitory computer-readable storage medium, or combinations thereof. The controller 444 may fetch, decode, and execute instructions, as described herein. As an alternative or in addition to retrieving and executing instructions, the controller may include at least one integrated circuit (IC), other control logic, other electronic circuits, or combinations thereof that include a number of electronic components for performing the functionality of instructions.

FIGS. 5A-5C illustrate other example apparatuses including sensor circuitry and a controller, consistent with the present disclosure. The apparatus 540 of FIG. 5A may include an implementation of and/or include substantially the same features and components of the apparatus 440 of FIG. 4, and is numbered accordingly. For instance, the apparatus 540 includes sensor circuitry 542 coupled to an interrogation region 511 of a microfluidic device 500 and a controller 544. The microfluidic device 500 may include an implementation of and/or include substantially the same features as the microfluidic device 100 of FIGS. 1A-1B and/or of FIGS. 3A-3G, such as including a microfluidic channel 502 coupled to a reservoir 503 and including an interrogation region 511 and a fluid ejection device 510 including a nozzle 507 and a fluid actuator 509.

In some examples, the sensor circuitry 542 includes sense circuits 543-1, 543-2 which may couple to the first sensor 504 and the second sensor 506 of the microfluidic device 500. For example, each of the first sensor 504 and the second sensor 506 may include a pair of sensing electrodes, with one electrode of the pair being grounded and another electrode of the pair coupled to a component of the sense circuits 543-1, 543-2. The first sensor 504 and the second sensor 506 are coupled to the sense circuits 543-1, 543-2 to provide an electric field between each respective pair of sensing electrodes. Fluid containing the particle is conductive, such as a phosphate buffered saline. As the particle flows through the electric field, an impedance-based or capacitance-based measure is obtained as a sensor signal by the first sensor 504 and the second sensor 506 and the coupled sense circuits 543-1, 543-2. For example, the sense circuits 543-1, 543-2 apply a voltage or current to one sensing electrode of the pair, with the other sensing electrode being grounded, and which causes the electric field to be applied within the microfluidic channel 502. As a particle flows by, a change in impedance or capacitance is measured by the sense circuits 543-1, 543-2, with the change being dependent on an altitude position of the particle within the microfluidic channel 502. For example, the closer the particle is to the sense circuits 543-1, 543-2, the greater the change in capacitance. Conversely, the further the particle is to the sense circuits 543-1, 543-2, the smaller the difference in capacitance.

The sensor circuitry 542 further includes an alternating current source 525 to apply an electrical signal (such as an alternating current signal) to the DEP actuator 508, which causes the non-uniform electric field to be applied within the microfluidic channel 502. As previously described, the DEP actuator 508 may include a DEP electrode and a ground source, such as a common ground shared between the first sensor 504, the second sensor 506, and the DEP actuator 508. The non-uniform electric field causes the DEP force to be applied to a passing particle. As previously described, depending on the frequency of the DEP actuator 508 and particle properties, the particle is either deflected away from or pulled toward the DEP actuator 508 in response to the DEP force. The two sensors 504, 506 are used to measure the altitude and other information of the particle by measuring the impedance or capacitance change of the electrode pairs before and after the DEP force is applied.

Using a particular example, as a particle passes over the first sensor 504, the particle is at a first position relative to the bottom of the microfluidic channel 502. The particle passes over the DEP actuator 508 and experiences an nDEP from the non-uniform electric field, causing the particle to shift to a second position relative to the bottom of the microfluidic channel 502, which is farther away from the bottom of the microfluidic channel 502 than the first position. The particle then passes over the second sensor 506. The first sensor 504 and the second sensor 506 are used to obtain sensor signals indicative of impedance-based measure. For example, the first sensor 504 is used to obtain a first sensor signal and the second sensor 506 is used to obtain a second sensor signal, with the first and second sensor signals being indicative of a change in a capacitance measure. As previously shown by FIGS. 2C-2D, the sensor signals may include peaks that indicate the change in position, with the first sensor signal having a greater peak (e.g., a greater change in capacitance) than the second sensor signal due to the change in altitude position, in the example.

FIG. 5B illustrates a close-up view of an example sense circuit 543-1 of the sensor circuitry 542 of FIG. 5A. Referring to FIG. 5A, each of the sense circuits 543-1, 543-2 may be implemented as illustrated by FIG. 5B. Referring back to FIG. 5B, the sense circuit 543-1 includes a capacitor 562, a power source 560, a switch 563, a ground path, an analog-to-digital (A/D) circuit 564, and a field programming gate array (FPGA) 566 or other processing circuitry. When the switch 563 is in a closed position, the power source 560 charges the capacitor 562 which is coupled to an anode pad 557 of a first sensing electrode of the first sensor of the microfluidic device 500 and causes an electric field within the microfluidic channel of the microfluidic device 500. The capacitor 562 may be charged for a first period of time, and then the switch 563 is transitioned to an open position, and a measurement is obtained for a second period of time. The first and second periods of time may be at fixed intervals to obtain a plurality of sensor signals associated with a plurality of particles. The ground path is coupled to the cathode pad 559 of a second sensing electrode of the first sensor. In some examples, the cathode pad 559 is shared between the first sensor, the second sensor, and the DEP actuator. The discharge of the capacitor 562 is observed using the A/D circuit 564 and FPGA 566. For example, in response to the particle passing by, the capacitor 562 discharges, which is captured by and converted to a digital signal by the A/D circuit 564 and identified by the FPGA 566. The FPGA 566 may be in communication with or form part of the controller.

FIG. 5C illustrates another example apparatus 541 including sensor circuitry 542 and a controller 544, consistent with the present disclosure. The apparatus 541 of FIG. 5B may include an implementation of and/or include substantially the same features and components of the apparatus 540 of FIG. 5A, with the sensor circuitry 542 including one sensor circuit 543 that is coupled to both the first sensor 504 and the second sensor 506. The common features and components are not repeated. In such examples, the sense circuit 543 may be similar to the sense circuit 543-1 of FIG. 5B, with the capacitor 562 coupled to anode pads associated with both the first and second sensors 504, 506.

FIGS. 6A-6B illustrate other example apparatuses including a microfluidic device, sensor circuitry, and a controller, consistent with the present disclosure. The apparatus 670 of FIG. 6A may include an implementation of and/or include substantially the same features and components of the apparatus 540 of FIGS. 5A-5B, and is numbered accordingly. For instance, the apparatus 670 includes sensor circuitry coupled to a microfluidic device 600 and a controller 644. The microfluidic device 600 may include an implementation of and/or include substantially the same features as any of the microfluidic devices of FIGS. 1A-3G, with the common features and components not being repeated. As shown, the microfluidic device 600 may include a common cathode pad 659 used as a shared ground for each of the first and second sensors and the DEP actuator, with each of the first sensor, the second sensor, and DEP actuator having a separate anode pad 657-1, 657-2, 657-3.

As shown by FIG. 6A, an example apparatus 670 includes a fluid dispensing device 672 and a microfluidic device 600. The fluid dispensing device 672 includes a substrate transport assembly, a controller 644, and sense circuits 643-1, 643-2, the common components of which are not repeated. The substrate transport assembly may include a stage 679 coupled to one of the substrate 678 and the fluid dispensing device 672 to move a position of the substrate 678 with respect to the fluid dispensing device 672. The fluid dispensing device 672 may include additionally non-illustrated components, such as a mounting assembly and a power supply that provides power to the various electrical components of the fluid dispensing device 672 and the microfluidic device 600 mounted therein. The fluid dispensing device 672 may control a fluid ejection device of the microfluidic device 600 to dispense droplets of fluid to the substrate 678. The fluid dispensing device 672 may cause flow of a fluid from a reservoir, through a microfluidic channel, and to the fluid ejection device of the microfluidic device 600, and then cause the fluid ejection device (e.g., actuate a resistor in an ejection chamber) to eject a volume of the fluid from the fluid ejection device to a region of the substrate 678, such as to a plurality of regions of the substrate 678.

In some examples, the apparatus 670 includes the substrate 678. The substrate 678 may include different regions, such as wells of a well plate, with each region getting a particle or a particle population depending on determined particle properties. These dispense locations may be specific target regions on the substrate surface, such as cavities, microwells, channels, indentation into the substrate, or other regions of the substrate.

The various illustrated apparatuses may operate in different modes of operations. In an example first mode of operation, the controller 644 identifies a single particle, such as a single cell, classifies the particle, and then directs the stage 679 to position the substrate 678 under the fluid dispensing device 672 aligned with the nozzle of the fluid ejection device of the microfluidic device 600, and causes ejection of the particle into a particular region (e.g., well) of the substrate 678. The process is completed, and then the controller 644 may output a dispense map indicative of a number of particle(s) and/or particle type located in each region of the substrate. The dispense map may be output to external control circuitry, such as for further processing of the particles. In the first mode or another mode of operation, the controller 644 may control the position of the substrate 678 to eject a type or classification of particles into a region and to eject other classes of particles, debris or other waste to a junk region.

In another mode of operation, the controller 644 identifies a single particle and classifies the particle, and then directs the stage 679 to position the substrate 678 under the fluid dispensing device 672 and the particles are ejected into particular groups of regions (e.g., groups of wells) which are grouped by particle classification. The controller 644 may output a dispense map indicative of a number of particle(s) and/or particle type or classification located in each group of regions of the substrate 678.

FIG. 6B illustrates another example apparatus 671 including a fluid dispensing device 672 and a microfluidic device 600, consistent with the present disclosure. The apparatus 671 of FIG. 6B may include an implementation of and/or include substantially the same features and components of the apparatus 670 of FIG. 6A, with the sensor circuitry including one sensor circuit 643 that is coupled to both anode pads 657-1, 657-2 of the first sensor and the second sensor and the common cathode pad 659 that is shared by the first sensor, the second sensor, and the DEP actuator, as previously described. The common features and components are not repeated.

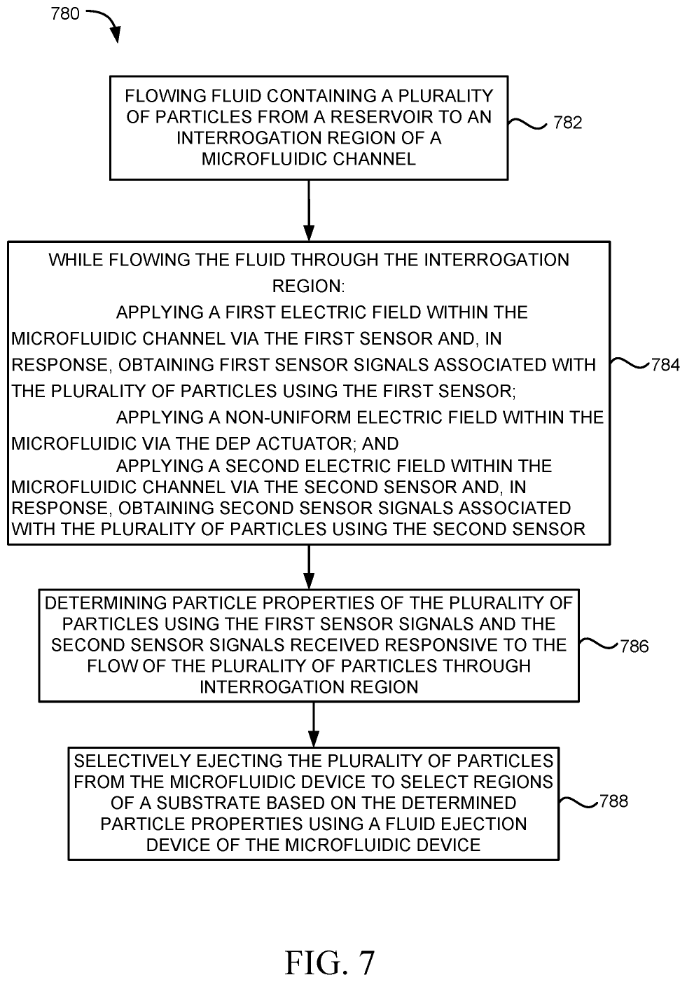

FIG. 7 illustrates an example method of selectively ejecting fluid using a microfluidic device, consistent with the present disclosure. The method 780 may be implemented by or using any of the microfluidic devices as illustrated by FIGS. 1A-3G and/or by the apparatuses of FIGS. 4-6.

At 782, the method 780 includes flowing fluid containing a plurality of particles from a reservoir to an interrogation region of a microfluidic channel of a microfluidic device. The fluid may be flowed at a constant flow rate, in some examples. As previously described, the interrogation region includes a first sensor disposed within the microfluidic channel, a second sensor disposed within the microfluidic channel, and a first DEP actuator disposed within the microfluidic channel between the first sensor and the second sensor.

At 784, while flowing the fluid through the interrogation region, the method 780 includes applying a first electric field within the microfluidic channel via the first sensor and, in response, obtaining a first sensor signal associated with the plurality of particles using the first sensor, applying a non-uniform electric field within the microfluidic via the DEP actuator, and applying a second electric field within the microfluidic channel via the second sensor and, in response, obtaining a second sensor signal associated with the plurality of particles using the second sensor.

In various examples, the first sensor signal and the second sensor signal may each include a continuous signal with a plurality of peaks associated with different particles traveling over the respective sensors. The continuous signals may be obtained over a total time which includes a plurality of measurement period of times and a plurality of capacitor charging periods of time. In other examples, the first sensor signal and the second sensor signal may include a plurality of first sensor signals and second sensor signals, with each of the plurality first sensor signals and second sensor signals being associated with measurements of one a plurality of particles traveling over the respective sensors.

At 786, the method 780 includes determining particle properties of the plurality of particles using the first sensor signal and the second sensor signal obtained responsive to the flow of the plurality of particles through interrogation region. As described above, determining the particle properties of the plurality of particles may include identifying a particle type based on at least one of signal shapes of the first sensor signal and the second sensor signal, peaks of the first sensor signal and the second sensor signal, and time between the peaks of the first sensor signal and the second sensor signal. For example, the particle properties may be selected from a particle size, a particle shape, a particle type, a particle flexibility, and a combination thereof, and determining the particle properties based on least one of a velocity, a shape of the first and second sensor signals, and a change in an altitude position.

At 788, the method 780 includes selectively ejecting the plurality of particles from the microfluidic device to select regions of a substrate based on the determined particle properties using a fluid ejection device of the microfluidic device. As previously described, a dispense map identifying classification of particles ejected and associated regions may be stored.

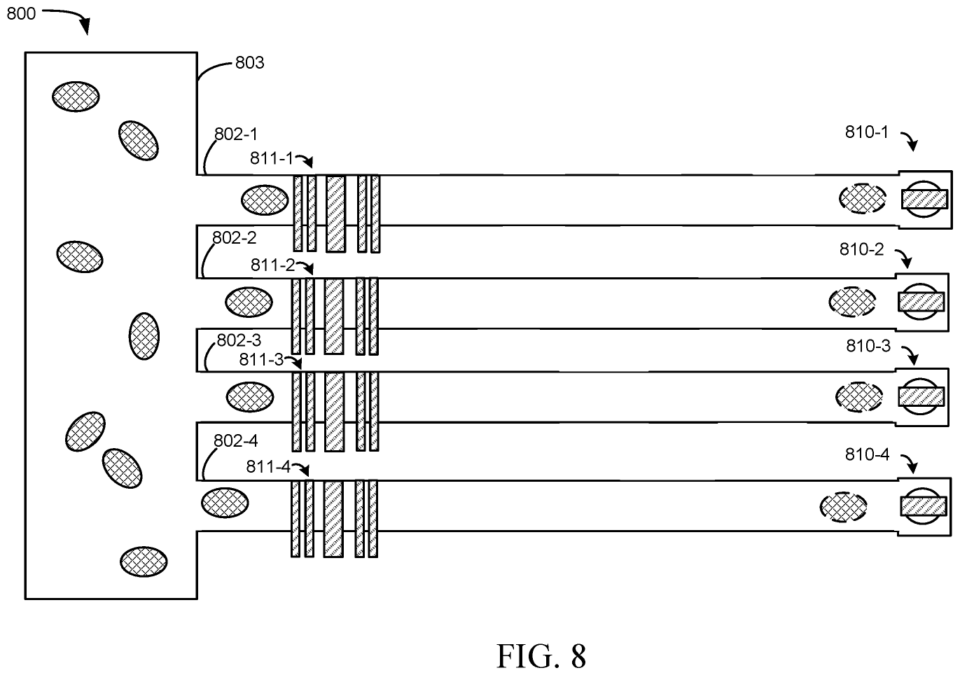

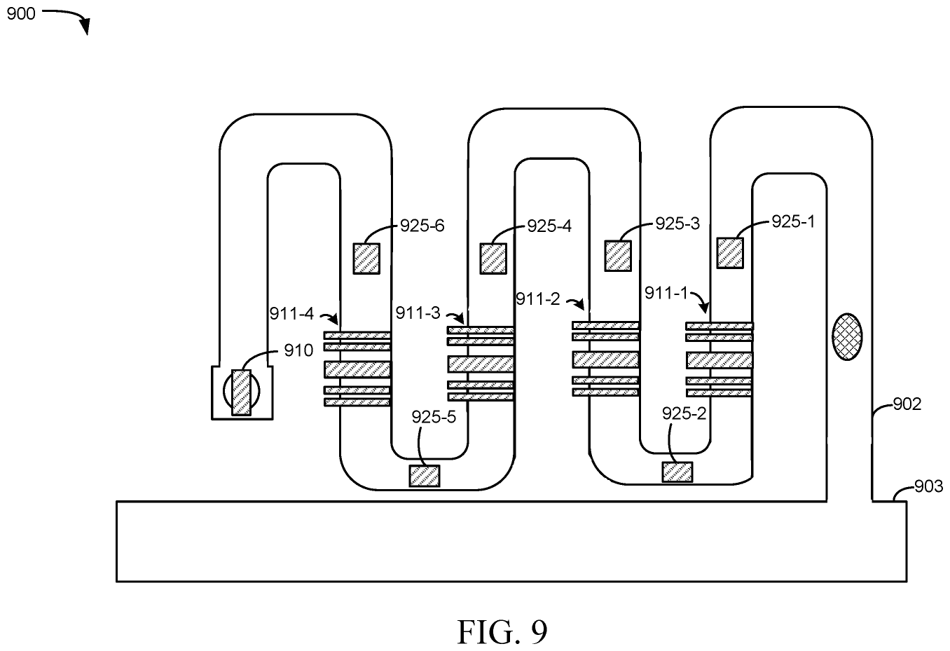

FIGS. 8-9 illustrates other variations that may be applied to any of the above-illustrated and described microfluidic devices and apparatuses.

FIG. 8 illustrates an example microfluidic device with a plurality of microfluidic channels and a plurality of interrogation regions, consistent with the present disclosure. The microfluidic device 800 includes substantially the same features and components as the microfluidic device 100 of FIGS. 1A-1B, with additional microfluidic channels 802-1, 802-2, 802-3, 802-4 coupled to the reservoir 803. Each microfluidic channels 802-1, 802-2, 802-3, 802-4 is fluidically coupled to a respective fluid ejection device 810-1, 810-2, 810-3, 810-4. Each fluid ejection device 810-1, 810-2, 810-3, 810-4 may dispense a volume of fluid into a different respective region of a substrate. The regions may be within a high-density micro-titer plate, such as a 1536 well plate, where the spacing between the nozzles of the fluid ejection devices 810-1, 810-2, 810-3, 810-4 match the spacing between different wells without moving the substrate. These channels may be similar and redundant, and used to increase throughput and/or to increase system robustness. The channels may be operated separately, concurrently, or in a round-robin fashion (cycling between which path is the active path). As previously described, each microfluidic channel 802-1, 802-2, 802-3, 802-4 includes an interrogation region 811-1, 811-2, 811-3, 811-4 including first and second sensors, and a DEP actuator, respectively, the common features and components of which are not repeated. A controller may control firing of each respective fluid ejection device 810-1, 810-2, 810-3, 810-4.

FIG. 9 illustrates an example microfluidic device with a microfluidic channel and a plurality of interrogation regions, consistent with the present disclosure. The microfluidic device 900 includes substantially the same features and components as the microfluidic device 100 of FIGS. 1A-1B, with additional interrogation regions 911-1, 911-2, 911-3, 911-4 along the microfluidic channel 902 fluidically coupled to a reservoir 903. The microfluidic channel 902 is not limited to the linear arrangement illustrated by FIGS. 1A-1B, and may include a curved arrangement fluidically coupled to a fluid ejection device 910 at an end. A plurality of fluid actuators 925-1, 925-2, 925-3, 925-4, 925-5, 925-6 may be disposed along the microfluidic channel 902 to provide the flow of fluid. Each interrogation region 911-1, 911-2, 911-3, 911-4 may include first and second sensors, and a DEP actuator, with the DEP actuators being fixed at different frequencies and/or otherwise adjustable to determine cross-over frequencies.

In some examples, the flow of fluid may be reduced in response to a particle being detected using sensor signals from one of the sensors of the first interrogation region 911-1. By reducing the flow of the fluid, the particle may move slower across subsequent interrogation regions 911-2, 911-3, 911-4 to allow for a more accurate measurement. The reduction in fluid flow may be performed by other microfluidic devices and/or apparatuses illustrated herein, such as the microfluidic devices 300, 330 of FIGS. 3A-3B, among others.

Any of the above illustrated microfluidic devices and apparatuses may include the variations illustrated herein in different combinations. The microfluidic devices and apparatuses may be used to determine particle properties and sort particles by classifying and distinguishing single particles from clumps or debris and/or identifying target particle types, such as sorting single cells. The cells may be sorted without using labels and which may reduce cell shearing risk as compared to other types of sensing. The electric properties determined may be used to identify alive cells verses dead cells, which may be useful for further cell analysis. For example, the above-described microfluidic devices and apparatuses may be used to eject higher live cell occupancy to a substrate, and without the use of labels, as compared to prior techniques.

Terms to exemplify orientation, such as left/right, and top/bottom, may be used herein to refer to relative positions of elements as shown in the figures. It should be understood that the terminology is used for notational convenience and that in actual use the disclosed structures may be oriented different from the orientation shown in the figures. Thus, the terms should not be construed in a limiting manner.

Various terminology as used in the Specification, including the claims, connote a plain meaning unless otherwise indicated. As examples, the Specification describes and/or illustrates aspects useful for implementing the claimed disclosure by way of various structure, such as controller, circuits, or circuitry selected or designed to carry out specific acts or functions, as may be recognized in the figures or the related discussion as depicted by or using terms such as device, and system, and/or other examples. Certain of these blocks may also be used in combination to exemplify how operational aspects have been designed and/or arranged. Whether alone or in combination with other such blocks or circuitry including discrete circuit elements such as resistors, these above-characterized blocks may be circuits coded by fixed design and/or by programmable circuitry for carrying out such operations. In certain examples, such a programmable circuitry includes and/or refers to computer circuits, including memory circuitry for storing and accessing a set of program code to be accessed/executed as instructions and/or data to perform the related operation. Depending on the data-processing application, such instructions and/or data may be for implementation in logic circuitry, with the instructions as may be stored in and accessible from a memory circuit. Such instructions may be stored in and accessible from a memory via a fixed circuitry, a limited group of configuration code, or instructions characterized by way of object code.

Where the Specification may make reference to a “first [type of structure]”, a “second [type of structure]”, etc., the adjectives “first” and “second” are not used to connote any description of the structure or to provide any substantive meaning; rather, such adjectives are merely used for English-language antecedence to differentiate one such similarly-named structure from another similarly-named structure designed or coded to perform or carry out the operation associated with the structure.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A microfluidic device comprising:
a microfluidic channel fluidically coupled to a reservoir containing a fluid;
a first sensor disposed within the microfluidic channel;
a second sensor disposed within the microfluidic channel;
a first dielectrophoretic (DEP) actuator disposed within the microfluidic channel between the first sensor and the second sensor; and
a fluid ejection device fluidically coupled to the microfluidic channel.

2. The microfluidic device of claim 1, wherein the fluid ejection device is to actuate to cause flow of the fluid from the reservoir and along the microfluidic channel such that a plurality of particles within the fluid pass over the first sensor, the DEP actuator, and the second sensor, and to eject a volume of the fluid from the microfluidic device.

3. The microfluidic device of claim 1, wherein the first sensor and the second sensor are impedance-based sensors that include a pair of sensing electrodes to provide impedance-based measures, and the DEP actuator includes a DEP electrode and a ground electrode.

4. The microfluidic device of claim 1, wherein the first sensor, the second sensor, and the DEP actuator share a common ground.

5. The microfluidic device of claim 1, wherein:
the first sensor is to provide a first electric field within the microfluidic channel and, in response, obtain a first sensor signal associated with a first particle within the fluid as the first particle passes by the first sensor in the microfluidic channel;
the DEP actuator is to provide a non-uniform electric field within the microfluidic channel as the first particle passes by the DEP actuator in the microfluidic channel; and
the second sensor is to provide a second electric field within the microfluidic channel and, in response, obtain a second sensor signal associated with the first particle as the first particle passes by the second sensor in the microfluidic channel.

6. The microfluidic device of claim 1, wherein the first sensor, the second sensor, and the DEP actuator form a first interrogation region between the reservoir and the fluid ejection device, and the microfluidic device further includes a second interrogation region between the first interrogation region and the fluid ejection device, the second interrogation region including:
a third sensor disposed within the microfluidic channel;
a fourth sensor disposed within the microfluidic channel; and
a second DEP actuator disposed within the microfluidic channel between the third sensor and the fourth sensor.

7. The microfluidic device of claim 1, further including:
a fluid actuator fluidically coupled to the microfluidic channel,
wherein the first sensor, the second sensor, and the DEP actuator are disposed between the fluid ejection device and the fluid actuator within the microfluidic channel.

8. The microfluidic device of claim 1, further including:
a second DEP actuator disposed within the microfluidic channel; and
a third sensor disposed within the microfluidic channel, wherein the second DEP actuator is disposed between the second sensor and the third sensor, and the first DEP is disposed on a first wall of the microfluidic channel and the second DEP actuator is disposed on a second wall of the microfluidic channel that is opposite the first wall.

9. An apparatus comprising:
sensor circuitry to couple to an interrogation region of a microfluidic channel of a microfluidic device and to obtain sensor signals using a first sensor and a second sensor, wherein the interrogation region includes:
the first sensor;
the second sensor; and
a dielectrophoretic (DEP) actuator disposed between the first sensor and the second sensor; and
a controller coupled to the sensor circuitry to:
cause flow of fluid including a particle through the interrogation region via actuation of a fluid ejection device coupled to the microfluidic channel;
apply electric fields within the microfluidic channel via the first sensor, the second sensor, and the DEP actuator, the electric fields including a non-uniform electric field applied via the DEP actuator;
determine a particle property of the particle using the sensor signals received from the sensor circuitry responsive to the flow of the particle through the interrogation region; and
cause the fluid ejection device to eject the particle from the microfluidic device to a select region of a substrate based on the determined particle property.

10. The apparatus of claim 9, further including the microfluidic device including the microfluidic channel and the fluid ejection device, wherein the sensor circuitry is coupled to the first sensor and the second sensor to obtain the sensor signals including:
a first sensor signal associated with the particle as the particle passes by the first sensor in the microfluidic channel; and
a second sensor signal associated with the particle as the particle passes by the second sensor in the microfluidic channel.

11. The apparatus of claim 10, wherein the controller and the sensor circuitry are to apply the electric fields including:
a first electric field applied via the first sensor and used to measure the first sensor signal in response;
the non-uniform electric field applied via the DEP actuator; and
a second electric field applied via the second sensor and used to measure the second sensor signal in response, wherein the controller is to determine the particle property based on a comparison between the first sensor signal and the second sensor signal responsive to the application of the non-uniform electric field.

12. The apparatus of claim 9, wherein the fluid includes a plurality of particles, the plurality of particles including the particle, and the controller is to:
determine particle properties of the plurality of particles;
cause the fluid ejection device to eject each of the plurality of particles from the microfluidic device to select regions of the substrate based on the determined particle properties; and
store a dispense map indicative of the select regions of the substrate that the plurality of particles are ejected to and as associated with the respective particle properties of the plurality of particles.

13. A method comprising:
flowing fluid containing a plurality of particles from a reservoir to an interrogation region of a microfluidic channel of a microfluidic device, the interrogation region including:
a first sensor disposed within the microfluidic channel;
a second sensor disposed within the microfluidic channel; and
a first dielectrophoretic (DEP) actuator disposed within the microfluidic channel between the first sensor and the second sensor;
while flowing the fluid through the interrogation region:
applying a first electric field within the microfluidic channel via the first sensor and, in response, obtaining a first sensor signal associated with the plurality of particles using the first sensor;
applying a non-uniform electric field within the microfluidic via the DEP actuator; and
applying a second electric field within the microfluidic channel via the second sensor and, in response, obtaining a second sensor signal associated with the plurality of particles using the second sensor;
determining particle properties of the plurality of particles using the first sensor signal and the second sensor signal obtained responsive to the flow of the plurality of particles through interrogation region; and
selectively ejecting the plurality of particles from the microfluidic device to select regions of a substrate based on the determined particle properties using a fluid ejection device of the microfluidic device.

14. The method of claim 13, wherein determining the particle properties of the plurality of particles includes identifying a particle type based on at least one of:
signal shapes of the first sensor signal and the second sensor signal;
peaks of the first sensor signal and the second sensor signal; and
time between the peaks of the first sensor signal and the second sensor signal.

15. The method of claim 13, wherein:
the particle properties are selected from a particle size, a particle shape, a particle type, a particle flexibility, and a combination thereof; and
determining the particle properties based on least one of a velocity, a shape of the first and second sensor signals, and a change in an altitude position.
